(19) United States
(12) Patent Application Publication
(10) Pub. No.: US 2006/0177610 A1
(43) Pub. Date: Aug. 10, 2006

(54) Sealing of Plastic Containers

(75) Inventors: Ian Gardner Cameron McAffer, Biggin Hill, Kent (GB); Peter Ernest Tasko, Herts (US)

Correspondence Address: STERNE, KESSLER, GOLDSTEIN & FOX PLLC, 1100 NEW YORK AVENUE, N.W., WASHINGTON, DC, 20005, US

(73) Assignee: Arrow International Limited, Valetta (MT)

(21) Appl. No.: 11/196,349

(22) Filed: Aug. 4, 2005

(51) Int. Cl. A61L 33/00; B31B 45/00; B28B 11/00

(52) U.S. Cl. 428/34.1; 427/2.1; 427/569; 428/34.4; 428/35.7

(57) ABSTRACT

Abstract of the Disclosure

An ampoule contains a solution, e.g. an inhalation or injectable pharmaceutical, and an outer surface of the ampoule is coated with a metal or metal compound so as to reduce moisture egress from the ampoule and reduce contamination of ampoule contents from external sources. Labels are easily applied to the coating.

### Detailed Description of the Invention

### FIELD OF THE INVENTION

The present invention relates to the sealing of containers, to the coating of containers made of plastics material used for pharmaceutical formulations, and in particular to coating ampoules to achieve a sealing effect. The invention relates also to the sealed or coated containers, in particular coated ampoules.

### BACKGROUND TO THE INVENTION

Pharmaceutical and cosmetic formulations are presented in a variety of different packaging, including packaging made of glass, metal, plastic and natural materials. For liquid formulations, e.g. solutions or suspensions, the packaging must be and remain sealed to prevent leakage. However, a number of technical and practical difficulties exist with all such containers.

Some formulations may contain highly volatile substances or other relatively small molecules that can diffuse out through the material of the container. This is a particular problem with, say, perfumes. Shelf-life is thus limited as products may lose potency, aroma or flavour. As a result, containers for such products are made of material that is impermeable e.g. glass, such materials being generally rather expensive. It is hence not possible to use cheaper materials such as plastics so high packaging costs are incurred.

Pharmaceutical formulations in containers may have to be sterilized under conditions of high temperature or pressure, or once filled under sterile conditions must be robust enough to maintain that sterility. Again, this tends towards higher production costs.

It is known to administer drugs to the lungs of a patient using a nebulizer, allowing a patient to administer the drug whilst breathing normally. The drugs are provided in a unit dose ampoule (UDA), containing a relatively small volume, typically 1mL - 5mL, of solution and typically made of plastics material. A method of making ampoules is by Blow-Fill-Seal (BFS), under aseptic conditions, in which the ampoule is formed by extrusion and filled with solution in a multi-part but essentially one-step process. If necessary, and provided the contents are not heat labile, heat sterilization can be used, e.g. ampoules can be sterilised by terminal sterilisation methods, i.e. after the ampoule has been filled and sealed. These methods are well established and accepted by regulatory authorities worldwide.

A known problem with existing ampoules is that they allow oxygen, other gases and other volatile compounds into the ampoule and allow water (moisture) to exit. Testing of the contents has revealed that, during storage, contaminants can pass through the plastic of ampoule walls and be absorbed into the formulation. As one specific example, unacceptable amounts of vanillin have been found inside ampoules, leading to failure of the product and refusal of regulatory authorities to licence the ampoules without safeguards against this external contamination.

The US FDA has recently required that ampoules be over-wrapped by a sealing pouch to avoid contamination of the ampoule contents. The pouch material is typically a tri-laminate of paper and/or polymer, aluminum and low density polyethylene (LDP). This pouch is regarded as an acceptable solution to the problem.

Ampoules are typically produced in strips of multiples of single units doses, e.g. fives, tens, thirties etc. Therefore, a problem with pouches is that if several ampoules are contained within one pouch then as soon as the pouch is opened and the first ampoule used, the remaining ampoules are exposed to the environment and can be contaminated.

The permeability of the LDP also restricts the labeling of the ampoules, as inks used for direct printing onto ampoules and adhesives used to attach paper labels must be checked carefully to ensure none will penetrate the ampoule and contaminate the contents.

Some ampoules are topped up with inert gas, e.g. nitrogen. Even in a pouch there is some equilibration of nitrogen with the gases outside the ampoule but inside the pouch. As soon as the pouch is opened more nitrogen will be lost from the ampoule.

LDP ampoules are translucent and some photo-sensitive materials when stored in these might be damaged after long-term storage and exposure to light. Pouches offer a partial solution but, again, once the pouch is opened ampoules inside are exposed to light for indefinite periods before being used.

Separately, LDP tubes are fairly commonly used for cosmetics. But it is necessary to avoid oxygen getting into certain tube contents, e.g. if there are liposomes or other oxygen sensitive contents. LDP and other such materials are as a result not generally acceptable for manufacture of tubes for these cosmetics.

An object of the present invention is to solve or at least ameliorate the above-identified issues. An object of preferred embodiments of the invention is to provide alternative, more preferably improved methods of sealing of containers, and containers, in particular ampoules sealed by the methods.

### SUMMARY OF THE INVENTION

The invention is based upon use of a metal-containing sealing layer to provide a coating on containers made of plastics material.

In a first aspect, the invention provides an ampoule, comprising a coating of a metal or a metal compound.

Generally, the invention provides a container for containing liquids, made of plastics material and comprising a coating of metal or a metal compound.

In a second aspect, the invention provides a method of reducing moisture egress from a container made of plastics material, comprising applying to an outer surface of the container a coating comprising a metal or a metal compound.

In a third aspect, the invention provides a method of sealing a container made of plastics material, comprising applying to an outer surface of the container a coating comprising a metal or a metal compound.

A fourth aspect of the invention provides a method of applying a label to an ampoule, comprising applying a coating of a metal or a metal compound to the ampoule and applying the label to the coating.

The coating can be applied by first providing the plastics layer and then applying the coating onto the layer or by producing, for example by extrusion or otherwise, a plastics layer coated with the metal coating.

### DETAILED DESCRIPTION OF THE INVENTION

A coated container of the invention is an ampoule having a coating of a metal or a metal compound. In use this coating is found to have the effect of sealing the contents of the ampoule, reducing loss of ampoule contents to the outside and reducing contamination of the contents from the outside.

The ampoule is typically of plastics material, especially polypropylene or polyethylene, low or high density or other polymer used in manufacture of ampoules or in the drinks industry, e.g. polyethylene terephthalate. Further, the ampoule will typically contain a pharmaceutical agent, such as an inhalation drug or injectable drug, in combination with a pharmaceutically acceptable carrier.

The sealing is not required to be complete but is preferred to be such that after testing for the periods required e.g. in the case of ampoules to satisfy the regulatory authorities that the contents are adequately protected so that no further steps such as provision of external overwrapping by pouches are imposed. The coating may hence cover at least 50% of the outer surface area of the ampoule, or at least 70%, 80%, 90% or 95% of the outer surface area of the ampoule. Very preferably substantially all of the outside of the ampoule is coated.

When a strip of ampoules is coated and one ampoule detached from the strip there may as a result be a side edge or portion of the remaining end ampoule which is uncoated and thus exposed, but this is likely to detract only slightly if at all from the overall sealing effect of the coating – the exposed portion being small compared to the total surface area and occurring at a position where the thickness of the plastic, the junction between adjacent ampoules, is generally greatest. The invention is thus useful for coating single containers or ampoules and also ampoules designed to be produced in strips and detached one-by-one.

The coating material can be selected from a wide variety of metals and metal compounds which can be coated onto e.g. the ampoule. The coating can comprise aluminium, copper, carbon, chromium, silver, zirconium, tantalum, tungsten, titanium, cobalt, gold, palladium, platinum, and their alloys, including steel, and their compounds, including compounds of metals with gases, for example carbon nitride, tin oxide, indium oxide, silicon dioxide. Some of these coating materials are more expensive than others and for containers such as ampoules made in large numbers and being essentially for once-only use the coating preferably comprises aluminium, titanium, chromium, silver, copper, or a mixture or alloy of the aforesaid. Particularly preferred coatings comprise or consist of aluminium, titanium, chromium or tetrahedral amorphous carbon.

To apply the coating, a number of different techniques may be employed. Suitable coating methods include physical vapour deposition, e.g. by sputtering, and arc deposition. Sputter coatings optionally have a UV lacquer to protect the coating and improve adhesion.

Sputtering deposition, as an example of physical vapour deposition, is performed in a vacuum chamber where atoms, generally argon atoms, are ionized and accelerated to strike a target material, say aluminium. Coating material enters the vapour phase through a physical process rather than by a chemical or thermal process. The argon atoms dislodge aluminium atoms when they strike the target, then these ejected aluminium atoms strike the container to be coated, and this process applies a dense coating. Argon (Ar) ions can be created in an ion gun which then imparts kinetic energy and directs the ions toward the target to be sputtered, or in a plasma that contains Ar+ and electrons. The plasma glows because of reactions between the electrons and atoms and ions and is neutral in charge. The spectral content of the glow is indicative of the ion species present and can be used to control the composition of the deposited film. The interactions between electrodes and ionized species and electrons are complicated, and the variety of sputtering configurations existent emphasize specific aspects of the plasma physics that is involved. For example, in magnetron sputtering powerful permanent magnets behind the target contain electrons in their fields to increase the probability of collisions with atoms and metastable species and thereby increase the density of available ions. In all forms of plasma sputtering, a virtual electrode is created at the boundary between the plasma and a volume known as the Crook's dark space, where electronic and ionic interactions are absent. Ar+ ions are extracted from the plasma and accelerated across the dark space to impinge on the target. During the momentum transfer at the target surface, positive and negative ions and electrons as well as atoms, dimers, and trimers are released. The positive ions return to the target where they contribute to heating. In some arrangements, negative ions and electrons can strike the substrate located near the anode.

Sputter rate is determined by target voltage and current density, as well as chamber pressure. High voltage and current (power) releases more sputtered species; high pressure provides more ion density but simultaneously reduces the energies of the ions and atoms by scatter. Each sputter process must be optimized for the materials used. It is generally held that sputtered films adhere better than evaporated films. The variety of materials from which sputtering targets can be made is nearly limitless. For example, alloys of materials having different evaporation pressures can be sputtered but not evaporated. Targets of single-element materials, such as metals, are generally the pure metal, while mixtures and doped composition targets are made by powder metallurgy. Powder mixtures are hot-pressed under appropriate atmosphere composition and may be sintered. Non-metal targets are made by ceramic technology. Multi-element (or compound) mixtures can be specially made.

Chemical vapour deposition or CVD is a generic name for a group of processes that involve depositing a solid material from a gaseous phase and is similar in some respects to physical vapour deposition (PVD). PVD differs in that the precursors are solid, with the material to be deposited being vaporised from a solid target and deposited onto the substrate. Whilst CVD may in some instances be suitable for the invention, generally the high temperatures required restrict the material that can be coated. CVD may also be too costly for large-scale manufacture of one-use products such as ampoules.

In arc deposition methods, an ion-containing plasma is created in a vacuum between an anode and a target, usually the cathode. In a filtered cathode arc, ions from the plasma are steered towards the substrate via a filter designed to remove neutral particles such as macroparticles. The ions deposit on the surface, forming the coating. The filtered vacuum cathode arc can apply coatings at lower temperatures, even lower than sputter coaters, below 70 degrees C and down to room temperatures, and is hence particularly suitable for temperature sensitive substrates such as plastics. Though, plastics which can withstand temperatures up to around 120 degrees C can be coated using sputter techniques. Metal or carbon or alloy coatings can be made using the filtered cathode arc, also compounds using introduction of reactive gas into the coating chamber near the substrate.

Examples of background reading on thin film technology including physical vapour deposition and vacuum arc deposition can be found in John A. Thornton and D. W. Hoffman, Thin Solid Films, 171, 5 (1989); J. Vossen and W. Kern, eds., Thin Film Processes, Academic Press, N. Y., 1978 and Handbook of Vacuum Arc Science and Technology by: Boxman, R.L.; Sanders, D.; Martin, P.J. © 1995 William Andrew Publishing/Noyes. Sputter apparatus is available from a number of commercial sources, including CPFilms Inc. of Martinsville, USA. FCVA Apparatus is also available from a number of commercial sources, including Nanofilm Technologies International Pte. Ltd of Singapore. Filtered cathode vacuum arc technology is described further in US patents 6,761,805, 6,736,949, 6,413,387 and 6,031,239, the contents of which are incorporated herein by reference. For the present invention, it is preferred that the coating is applied by physical vapour deposition or arc deposition.

Prior to coating of articles it is often preferred to carry out cleaning or other preparation of the surface, to remove contaminants and improve the adherence of the coating. For the containers of the invention aqueous cleaning is generally sufficient and can be omitted. For embodiments of the invention in which the articles to be coated is made of or comprises polymer such articles can be cleaned using known procedures except that more careful handling may be required. In addition, during aqueous cleaning polymers may absorb water which must later be removed to achieve vacuum coating adhesion. The coating may adhere without any treatment in which case even aqueous washing can be omitted.

The articles will likely remain clean for only a short period unless in a special environment, such as a dry nitrogen-purged container or in a UV/ozone chamber. One option is to provide a cleaning and/or surface preparation station as part or in juxtaposition to the coating station. A further consideration is that newly formed or moulded polymer, as in the blow-fill-seal process used for ampoule formation may not require any surface preparation for adequate adhesion of the coating to be obtained.

In use of the invention, ampoules can be prepared by forming the ampoule and applying the coating to the ampoule. A known method of forming ampoules is by blow-fill-seal (BFS), and the coating step can conveniently be added to the ampoule production line immediately after the BFS step and prior to packaging and/or labeling. The ampoules typically contain from about 1mL to about 5mL (extractable volume) of solution.

The coating is designed to achieve sealing of the containers, as described above. A suitable depth is of at least 20 nm, preferably at least 50 nm, and also suitably up to 50 microns, preferably up to 20 microns. The coating depth may also be at least 100 nm and up to 10 microns.

In a specific embodiment of the invention, an ampoule is made of plastics material and comprises a coating of aluminium applied by sputter coating. More specifically, the ampoule contains a solution of an inhalation pharmaceutical in a pharmaceutically acceptable carrier. Thus, in an example of the invention in use, blow-fill-seal technology is used to obtain ampoules containing 3ml of a formulation containing a salbutamol solution. The ampoules are made from LDP and exit the filling apparatus in strips of 10. The strips are coated with an external coating of aluminium, applied using a sputter coater, to a depth of approximately 300 nm, giving a shiny metallic look. The ampoules are packaged in the usual way though not overwrapped. Patients are given the ampoules in strips and tear off one ampoule at a time. The remaining ampoules are kept in a (now reduced size) strip until the next ampoule is removed and used, and so on until all ampoules are used.

In further embodiments of the invention, an ampoule is made of plastics material, comprises a coating of aluminium, chromium or titanium applied by sputter coating or filtered cathode arc and contains a solution of an injectable pharmaceutical in a pharmaceutically acceptable carrier. The solution may for example be water for injection or saline for injection. Typical volumes are 30ml or less, 25ml or less, 20ml or less, 15ml or less or 10ml or less. The ampoules can be manufactured in strips of 5, 10, 15 or more, as for other embodiments of the invention, to be torn off and used when required.

In a further specific embodiment of the invention, a plastic ampoule is coated with a layer of titanium, applied by sputter coating, to a depth of about 150 nm.

In a further specific embodiment of the invention, a plastic ampoule is coated with tetrahedral amorphous carbon to a depth of about 100 nm.

Whilst embodiments of the invention have been described with reference to coatings applied to ampoules, the invention in certain embodiments relates more generally to containers for containing liquids, made of plastics material and comprising a coating of metal or a metal compound. These containers can be made of polymer comprising polyethylene or polypropylene and further can have a maximum filled volume of up to 100ml, preferably up to 50ml, more preferably up to 20ml. The containers are useful for liquids containing volatile substances which would otherwise permeate plastics containers to an unacceptable degree.

Also provided by the present invention are a method of reducing moisture egress from a container made of plastics material, comprising applying to an outer surface of the container a coating comprising a metal or a metal compound, and a method of sealing a container made of plastics material, comprising applying to an outer surface of the container a coating comprising a metal or a metal compound. In these methods, the coating and its application are as described with respect to the above embodiments of the invention.

A further specific method of the invention is for sealing an ampoule, wherein the ampoule comprises from 0.5ml to 10ml of an inhalation pharmaceutical or an injectable pharmaceutical (e.g. water or saline for injection) in a pharmaceutically acceptable carrier, comprising applying to the ampoule a coating of a metal or a metal compound over at least 70% of the outer surface of the ampoule.

The coating of the invention has an additional or alternative property, naming that a label can be applied onto the coating. Hence, a further method of the invention is a method of applying a label to an ampoule, comprising applying a coating of a metal or a metal compound to the ampoule and applying the label to the coating.

The label can be attached to the coated ampoule using adhesive. The label can also be sprayed or printed onto the coated ampoule.

The inventions in its varying embodiments has a number of advantages, some or several or all of which may be seen in any given embodiment. The ampoules are sealed by the invention; reducing the loss e.g. of moisture and reducing contamination from the outside. Because of the shape of the ampoules, the process effectively seals each ampoule individually although ampoules may still be made in strips of say 5, 10, 30 etc. This is an improvement upon packaging a strip of ampoules in a pouch, as now when an ampoule is removed from the strip the remaining ampoules remain substantially sealed – contrast this with when a pouch containing many ampoules is opened and all become exposed to the environment.

Post application of the coating, it is relatively easy to apply labels to the ampoules or print with conventional inks, without the constraints upon choice of ink or presence of solvent that applied previously.

Ampoules coated according to the invention with a metallic coating have, in addition, a striking appearance. The coating has been found to be continuous, non-flaky and resistant to abrasion such as rubbing.

In relation to aspects of the invention in which other plastic containers are coated, the method applies generally to packaging used where the contents would be damaged by loss of or contamination by gases and other volatiles, for example, vitamins, flavours, perfumes etc. The invention provides packaging which is of plastics material, e.g. LDP, and cheaper than glass, trilaminates, ceramics etc.

The invention is now illustrated in the following examples, with reference to the accompanying drawings, in which:-

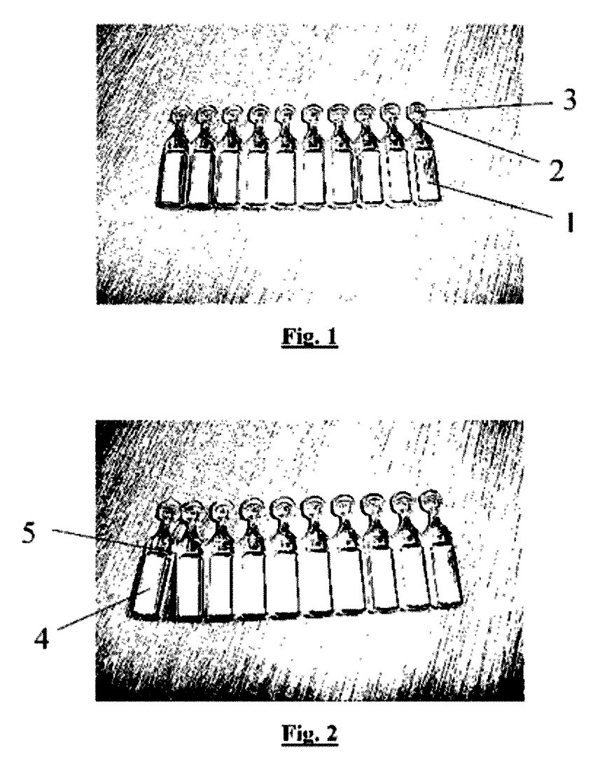

Fig. 1 shows a view from the front of a strip of ten ampoules coated with aluminium according to the invention;

Figs. 2 and 3 shows the strip of Fig. 1 with one ampoule being detached; and

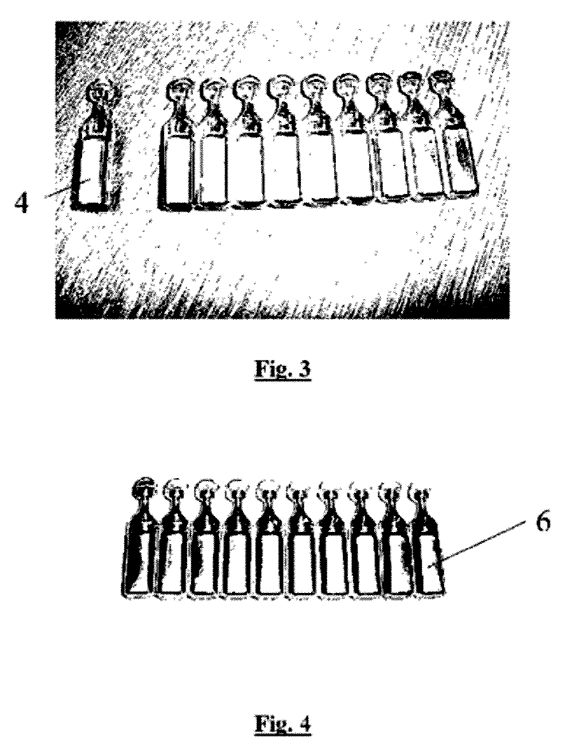

Fig. 4 shows a view from the front of a strip of ten ampoules coated with titanium according to the invention.

EXAMPLES

Example 1

A strip of 10 ampoules made from low density polyethylene was prepared using a standard blow-fill-seal apparatus, each ampoule containing 3ml of salbutamol solution. The ampoules were inspected visually to confirm correct filling of contents and manually to confirm they were all intact. The strip of ampoules was introduced into a filtered cathode arc coating machine fitted with an aluminium target. The machine was closed and pumped down to operating vacuum. The coating operation was begun and continued until the coating thickness monitor indicated a thickness of 300 nm. The coating was stopped, the vacuum released and the chamber opened.

The coated ampoules (1) are shown in Figures 1-3. The ten ampoules exited the coating chamber intact – Fig. 1 and have a head (3) which in use is twisted to break the neck (2) to release the contents.

The resultant coated ampoules had a shiny, metallic appearance, being completely coated with a thin layer of aluminum.

The aluminum coating was continuous over the whole surface of the ampoules, was smooth and without noticeable defects. The coating was firmly adhered to the ampoules and did not detach and resisted rubbing.

A single ampoule (4) was detached from the strip of 10 - See Fig. 2 – without tearing of the coating at the junction (5) between the detached ampoule and the remaining strip of nine ampoules.

The integrity of the ampoules was tested and it was confirmed they remained intact and contained the same volume of solution as prior to being coated. The contents of four ampoules were tested independently using an atomic absorption based method to determine whether there had been contamination by aluminium. In each separate test, an aluminium content of less than 1 ppm was recorded, beyond the lower limit of the detection method, confirming that the aluminium content of the solution inside the ampoule after coating was nil in each case. These results confirmed that the ampoule wall had not been breached during the coating process.

Example 2

A strip of 5 ampoules was made from low density polyethylene using a standard blow-fill-seal apparatus, each ampoule containing 3ml of saline solution. The ampoules were inspected visually to confirm correct filling of contents and manually to confirm they were all intact.

The strip of ampoules was introduced into a filtered cathode vacuum arc apparatus fitted with a titanium target. The machine was closed and pumped down to operating vacuum. The coating operation was begun and continued until the coating thickness monitor indicates a thickness of 300 nm. The coating was stopped, the vacuum released and the chamber opened.

The resultant coated ampoules (6) are shown in Fig. 4 and were found to have a shiny appearance, being substantially completely coated with a thin layer of titanium, the coating being slightly duller than the aluminum coating of Example 1.

The invention hence provides coated plastic containers and methods of obtaining the same.

What is Claimed is:

1. An ampoule, comprising a coating of a metal or a metal compound.

2. The ampoule of claim 1, made of plastics material.

3. The ampoule of claim 1, wherein the coating covers at least 50% of the outer surface area of the ampoule.

4. The ampoule of claim 3, wherein the coating covers at least 70% of the outer surface area of the ampoule.

5. The ampoule of claim 1, wherein the coating is selected from the group consisting of aluminium, titanium, chromium, silver, copper, tetrahedral amorphous carbon and mixtures and alloys of the aforesaid.

6. The ampoule of claim 1, wherein the coating is applied by physical vapour deposition or arc deposition.

7. The ampoule of claim 1, made by forming the ampoule and applying the coating to the ampoule.

8. The ampoule of claim 1, wherein the coating is a thin film of metal.

9. The ampoule of claim 8, wherein the coating has a depth of at least 20 nm.

10. The ampoule of claim 9, wherein the coating has a depth of up to 20 microns.

11. The ampoule of claim 1 made of plastics material and comprising a coating of aluminium.

12. The ampoule of claims 11, wherein the ampoule contains a solution of an inhalation pharmaceutical or an injection pharmaceutical in a pharmaceutically acceptable carrier.

13. The ampoule of claim 1, made of plastics material and comprising a coating of titanium.

14. The ampoule of claim 13, wherein the ampoule contains a solution of an inhalation pharmaceutical or an injection pharmaceutical in a pharmaceutically acceptable carrier.

15. The ampoule of claim 1, made of plastics material and comprising a coating of chromium.

16. The ampoule of claim 15, wherein the ampoule contains a solution of an inhalation pharmaceutical or an injection pharmaceutical in a pharmaceutically acceptable carrier.

17. A method of reducing moisture egress from a container made of plastics material, comprising applying to an outer surface of the container a coating comprising a metal or a metal compound.

18. The method of claim 17, comprising applying the coating over at least 50% of the outer surface of the container.

19. The method of claim 17, comprising applying the coating by physical vapour deposition or arc deposition.

20. The method of claim 17, wherein the metal is selected from the group consisting of aluminium, titanium, chromium and tetrahedral amorphous carbon.

21. A method of sealing an ampoule, wherein the ampoule comprises from 0.5ml to 10ml of an inhalation pharmaceutical in a pharmaceutically acceptable carrier, comprising applying to the ampoule a coating of a metal or a metal compound over at least 70% of the outer surface of the ampoule.

22. A method of applying a label to an ampoule, comprising applying a coating of a metal or a metal compound to the ampoule and applying the label to the coating.

23. The method of claim 22, wherein the label is attached to the coated ampoule using adhesive.

24. The method of claim 22, wherein the label is sprayed or printed onto the coated ampoule.

25. An ampoule made of plastics material, containing up to 50ml of a solution of an inhalation pharmaceutical, wherein the ampoule is coated with a metal or metal compound.

26. An ampoule made of plastics material, containing up to 50ml of a solution of an injectable pharmaceutical, wherein the ampoule is coated with a metal or metal compound.
